(19) United States
(12) Patent Application Publication
(10) Pub. No.: US 2023/0309815 A1
(43) Pub. Date: Oct. 5, 2023

(54) VISION TESTING DEVICE, VISION TESTING SYSTEM, AND VISION TESTING PROGRAM

(71) Applicant: CREWT MEDICAL SYSTEMS, INC., Tokyo (JP)

(72) Inventors: Satoshi INOUE, Tokyo (JP); Shinji KIMURA, Tokyo (JP); Tetsuya EGUCHI, Tokyo (JP)

(73) Assignee: CREWT MEDICAL SYSTEMS, INC., Tokyo (JP)

(21) Appl. No.: 18/018,797

(22) PCT Filed: Jan. 26, 2021
(86) PCT No.: PCT/JP2021/002653

§ 371 Date: Jan. 30, 2023

(30) Foreign Application Priority Data
Jul. 29, 2020 (JP) 2020-127947

(51) Int. Cl. A61B 3/024; A61B 3/00

(52) U.S. Cl. CPC A61B 3/024; A61B 3/0091; A61B 3/0033; A61B 3/0041

(57) ABSTRACT

A vision testing device and related technique, the vision testing device including a touch screen, sequentially displaying visual targets on the touch screen while displaying a fixation target whose position is fixed on the touch screen, during a period from the beginning to the end of the test, and providing a test result based on whether a testee taps the visual target or not.

### TECHNICAL FIELD

The present disclosure relates to a vision testing device, a vision testing system, and a vision testing program.

### BACKGROUND ART

A so-called visual field test which tests for visual field defects in glaucoma is known as one of vision tests. For example, as in Patent Document 1, there is a known vision testing device that uses an HMD and gives a response using a response switch. Also, as in Patent Document 2, there is a known technique for monitoring visual field defects in glaucoma using a touch screen.

### PRIOR ART DOCUMENT

[Patent Document]
Patent Document 1: Japanese Patent Laid-Open Publication No. 2017-000224
Patent Document 2: Japanese Translation of PCT International Application Publication No. 2015-502238

### SUMMARY OF DISCLOSURE

### Problems to be Solved by Disclosure

According to the description in Patent Document 2, during a period from the beginning to the end of the test, the system selects a target displayed on a touch screen, sets the target as a fixation target after a testee catches the target, then selects another target, and repeats such operations to perform a test. That is, dynamic fixation is employed in which a fixation target is not fixed to a single point on the touch screen (paragraphs 0024, 0054, etc.).

When the dynamic fixation is employed, the test results must be transformed into a case where the initial location of the gaze of the subject is at one location (e.g., center) on the touch screen in order to determine the state of the visual field defects. Further, the target must be arranged at a predetermined location on the touch screen in advance, taking into account such transformation.

The main object of the present disclosure is to provide a technique for performing a vision test without performing transformation processing on the test results using the dynamic fixation.

### Solution to Problem

A first aspect of the present disclosure provides a vision testing device including a touch screen, sequentially displaying visual targets on the touch screen while displaying a fixation target whose position is fixed on the touch screen, during a period from the beginning to the end of the test, and providing a test result based on whether a testee taps the visual target or not.

In a second aspect that is an aspect of the first aspect, multiple rounds of presenting the visual target to the testee include a round where the number of visual targets to be presented at once is more than one, during a period from the beginning to the end of the test, and the testee tapping all of the visual targets presented is considered as correct.

A third aspect that is an aspect of the first or second aspect, further including a mark for standby position of a hand of the testee to prevent the hand from remaining positioned between the testee and the touch screen.

A fourth aspect provides a vision testing system sequentially displaying visual targets on a touch screen while displaying a fixation target whose position is fixed on the touch screen, during a period from the beginning to the end of the test, and providing a test result based on whether a testee taps the visual target or not.

A fifth aspect provides a vision testing program for making a computer function to sequentially display visual targets on a touch screen while displaying a fixation target whose position is fixed on the touch screen, during a test, and to provide a test result based on whether a testee taps the visual target or not.

Other aspects that can be combined with each of the above-described aspects are as follows.

The number of visual targets displayed at once on the touch screen may be one or more than one. The number of visual targets to be displayed at once may be varied, in at least one round of multiple rounds of presenting the visual target to the testee, during a period from the beginning to the end of the test. On the other hand, during the period from the beginning to the end of the test, the number of visual targets may remain one, or may remain more than one. Alternatively, during the period from the beginning to the end of the test, the number of visual targets may be varied, for example, the number of visual targets may be one for the first round of presenting the visual target, two for the second round, and three for the third round. These patterns may be combined. When more than one visual targets are presented to the testee, the testee tapping all of the visual targets presented is preferably considered as correct.

Tap sensitivity of the visual target may be varied depending on eccentricity of the visual target from the center of the touch screen.

For example, when a tap judgment function is also imparted to the pixels surrounding the visual target, extended from the visual target to which the tap judgment function has been imparted, as the eccentricity is larger, the degree of extension may be increased.

In order to increase the tap sensitivity, the tap judgment function imparted to the pixels of the visual target may be extended and imparted to the pixels surrounding the visual target as well. As the number of pixels from the pixel at the center of the touch screen to the pixels of the visual target is larger, the degree of extension at that time may be increased.

As a specific aspect of increasing the degree of extension, a range to which the tap judgment function is imparted may be defined by the number of pixels from the pixels of the visual target. For example, the number of pixels from the pixel at the center of the touch screen is classified into three levels: low, medium, and high. In a case of “low”, the tap judgement function is imparted only to the pixels of the visual target; in a case of “middle”, the tap judgement function may also be imparted to a single circle of pixels surrounding the pixels of the visual target; and in a case of “high”, the tap judgement function may also be imparted to a double circle of pixels surrounding the pixels of the visual target. The present disclosure is not limited to this aspect, and the number of the extended pixels is not limited.

An example using numerical values will be described. It is desirable that, in a case where the number of pixels corresponds to the eccentricity (visual angle) of 20°, an area of the extended pixels to which the tap judgment function is imparted is about 4 times the area in a case where the number of pixels corresponds to the eccentricity (visual angle) of 10°, in consideration of the relation between the eccentricity (visual angle) and the visual acuity.

The vision testing device may include a calculation unit that calculates a degree of variation in the tap sensitivity depending on the eccentricity of the visual target. The calculation unit may utilize a calculation function of the known tablet device. The calculation function may be controlled by a control unit of the known tablet device.

The vision testing device may include a tap sensitivity regulating unit that reflects the degree of variation in the tap sensitivity onto the touch screen. The tap sensitivity regulating unit and the calculation unit may have common configuration. The tap sensitivity function may be controlled by the control unit of the known tablet device.

Attention level of the visual target may be varied depending on the eccentricity of the visual target from the center of the touch screen.

The “attention level of the visual target” means a degree of ease for the testee to recognize the visual target. “To vary the attention level of the visual target” means to vary the degree of ease for the testee to recognize the visual target.

As the eccentricity is larger, increasing the attention level of the visual target, thereby increasing contrast, luminance, or size of the visual target, or increasing presentation time of the visual target, or any combination thereof may increase the attention level of the visual target.

Specific examples of varying the attention level of a visual target include increasing the contrast, luminance, or size of the visual target, or increasing the presentation time of the visual target, or any combination thereof.

In a case of increasing the luminance of the visual target to increase the attention level of the visual target, the luminance of the visual target B with medium eccentricity is increased compared to the visual target A with low eccentricity, as shown in FIG. 3. Similarly, the luminance of the visual target C with high eccentricity is increased compared to the visual target B with medium eccentricity.

An example using numerical values will be described. Regarding contrast and luminance, the contrast in a case where the eccentricity is 20 pixels is desirably higher than the contrast in a case where the eccentricity is 10 pixels by about 6 dB±4 dB (2 dB to 10 dB), in consideration of the relation between the eccentricity and the sense of human.

Regarding presentation time, the presentation time in a case where the eccentricity is 20 pixels is desirably longer than the presentation time in a case where the eccentricity is 10 pixels by about 2±0.5 times (1.5 to 2.5 times).

In a case of increasing the contrast and/or size of the visual target, which is another specific example of increasing the attention level of the visual target, and/or in a case of increasing the presentation time of the visual target, the “luminance” in the above paragraphs may be replaced with the “contrast, size, and/or presentation time of the visual target”.

The vision testing device may include a calculation unit that calculates a degree of variation in the attention level depending on the eccentricity of the visual target. The calculation unit may utilize a calculation function of the known tablet device. The calculation function may be controlled by the control unit of the known tablet device.

The vision testing device may include an attention level regulating unit that reflects the degree of variation in the attention level onto the touch screen. The attention level regulating unit and the calculation unit may have common configuration. An attention level function may be controlled by the control unit of the known tablet device.

The tablet device, which is a computer, may include a display unit, an input unit, and a control unit. It is preferred that the control unit executes a predetermined program, so that the tablet device functions as a fixation target display unit, a visual target display unit, a tapping detection unit, a calculation unit, and a regulating unit (for the tap sensitivity, attention level regulating unit, and/or response time limit).

It is better for the vision testing device to include a touch screen that presents a visual target to a testee while presenting a fixation target whose position is fixed on the touch screen, and lets the testee tap the visual target within a response time limit, during the period from the beginning to the end of the test, where the response time limit given to the testee may be variable for each round of the multiple rounds of presenting the visual target to the testee during the period from the beginning to the end of the test.

The response time limit may be variable depending on the number of visual targets presented at once in each round of the multiple rounds of presenting the visual target to the testee, during the period from the beginning to the end of the test.

It is preferable that a round where the number of visual targets to be presented at once is more than one is included, and that the testee tapping all of the visual targets presented is considered as correct.

The above-mentioned response time limit may be variable depending on the response time required by the testee in the past.

### Advantageous Effects of Disclosure

According to the present disclosure, the vision test can be performed without transformation processing of test results with the dynamic fixation.

### BRIEF DESCRIPTION OF DRAWINGS

FIG. 1 is a schematic diagram of a touch screen of a vision testing device of this embodiment displaying a visual target.

FIG. 2 is a diagram illustrating how to count the number of pixels from the pixel at the center of the touch screen of the vision testing device of this embodiment.

FIG. 3 is a diagram illustrating the arrangement of the visual target and a region to which the tap judgment function is imparted when the eccentricity of the visual target of the vision testing device of this embodiment is classified into three levels: low (FIG. 3(a)), medium (FIG. 3(b)), and high (FIG. 3(c)).

FIG. 4 is a diagram illustrating the difference in the luminance of the visual target when the eccentricity of the visual target of the vision testing device of this embodiment is classified into three levels: low (FIG. 4(a)), medium (FIG. 4(b)), and high (FIG. 4(c)).

FIG. 5 is an explanatory diagram illustrating how the response time limit is varied depending on the number of visual targets displayed at once in the vision testing device of this embodiment.

FIG. 6 is a block diagram including a configuration of a control system of the vision testing device of this embodiment.

FIG. 7(a) is a diagram illustrating a case where a hand of a testee remains positioned between the testee and the touch screen when the visual target is presented to the testee in the vision testing device of this embodiment, and FIG. 7(b) is a diagram illustrating a case where a mark for standby position of a hand is provided and the testee complies with the mark.

### DETAILED DESCRIPTION OF DISCLOSURE

Embodiments of the present disclosure will be described hereafter. In this embodiment, a case where the vision testing device is a perimeter is exemplified.

In this embodiment, a case where the vision testing device is a tablet device is described. The vision testing device of this embodiment includes a touch screen that presents a visual target to the testee and lets him/her tap the visual target. Such a touch screen includes a display surface of the tablet device.

The “visual target” described here is displayed to give a stimulus by light to an eyeball of the testee when testing a vision of the testee. With regard to the visual target, there is no particular limitation on size, shape, and the like. For example, during glaucoma test, it is possible to test (identify) presence or absence of a visual field defect and a location of the defect, by displaying a point of light in a predetermined size as a visual target and varying the position of the point of the light.

The tap judgment function is imparted to the pixels of the visual target. The “tap judgment function” is a function to judge as correct the testee tapping and pressing the visual target displayed on the touch screen, more specifically, the pixel portion displayed on the touch screen with higher luminance than that of the surroundings. The tap judgment function may be referred to as a collision detection function. The “correct” used herein means that the testee can recognize the visual target in a predetermined visual field. When the pixels of the visual target to which the tap judgment function has been imparted do not detect pressure within a specified time, the vision testing device judges it as incorrect. The “incorrect” used herein means that the testee cannot recognize the visual target in a predetermined visual field.

The number of visual targets displayed at once on the touch screen may be one or more than one. The number of visual targets to be displayed at once may be varied, in at least one round of the multiple rounds of presenting the visual target to the testee, during a period from the beginning to the end of the test. On the other hand, during the period from the beginning to the end of the test, the number of visual targets may remain one, or may remain more than one. Alternatively, during the period from the beginning to the end of the test, the number of visual targets may be varied, for example, the number of visual targets may be one for the first round of presenting the visual target, two for the second round, and three for the third round. When more than one visual targets are presented to the testee, it is preferable to have all of the more than one visual targets to be tapped.

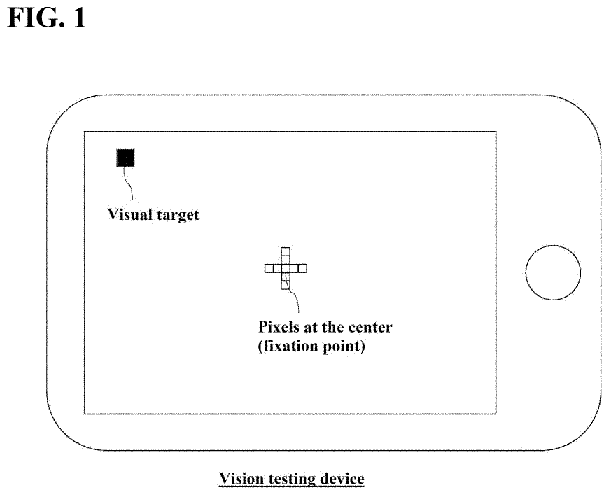

FIG. 1 is a schematic diagram of the touch screen of the vision testing device of this embodiment displaying a visual target.

When using tablet devices, existing tablet devices (smartphones, tablet computers, etc.) can be used for hardware functions. This embodiment may be performed by a program that regulates function related to the tap sensitivity of the tablet device.

Preferred examples of this embodiment are listed below.

### (Tap Sensitivity)

One of the characteristics of this embodiment is to vary the tap sensitivity of the visual target depending on the eccentricity of the visual target from the fixation target.

The pixel at the center of the touch screen is usually a pixel that constitutes the fixation target. During the period from the beginning to the end of the test, the visual targets are sequentially displayed on the touch screen while displaying a fixation target whose position is fixed on the touch screen (as an example, a case where the target is fixed at the center of the touch screen is exemplified hereafter, but other positions should not be excluded). The testee taps the visual target presented while fixating the fixation target. The test result is obtained based on whether the testee taps the target or not. The shape of the fixation target is arbitrary and may be a cross. For the sake of explanation, it is also shown as a dot in the present specification. The fixation target may be displayed constantly or may be displayed blinking on the touch screen. The phrase “while displaying the fixation target” encompasses both cases. Anyway, there is no limitation on the display mode of the fixation target as long as the testee can fixate the fixation target when tapping the visual target.

The phrase “eccentricity of the visual target from the center of the touch screen” means a degree of separation between the visual target and the center of the touch screen, from the center of the touch screen to the display limit of the touch screen or to the extreme end and farthest portion of the touch screen that is used for the vision test.

The term “eccentricity” may be expressed as the number (i.e., absolute number) of pixels from the pixel at the center of the touch screen to the pixels of the visual target (how to count the number of pixels will be described below), or may be expressed as a percentage of the number (i.e., relative number) of pixels from the center of the touch screen to the visual target with respect to the number of pixels from the center of the touch screen, through the visual target, to the extreme end and farthest portion of the touch screen that is used for the vision test. Hereafter, the eccentricity encompasses both of the case using the absolute number and the case using the relative number. Alternatively, the “eccentricity” may be expressed as an angle (i.e., viewing angle) between the line of sight to the pixel at the center of the touch screen and the line of sight to a predetermined pixel. In such a case, it is indicated as the eccentricity (visual angle).

When using the touch screen, for the testee, it is easy to touch the central portion of the touch screen while it is difficult to touch the end portion of the touch screen. In contrast, by adopting the present configuration, the difference in the tap sensitivity of the visual target on the touch screen between the central portion and the end portion can compensate for the difference in operability between the central portion and the end portion of the touch screen. As a result, the test accuracy when utilizing a touch screen can be improved.

A specific example of providing a difference in tap sensitivity between the central portion and the end portion will be described below. The present disclosure is not limited to the specific example described below.

For example, as the eccentricity is larger, the degree of extension may be increased when the tap judgment function imparted to the pixels of the visual target is extended and imparted to pixels surrounding the visual target as well.

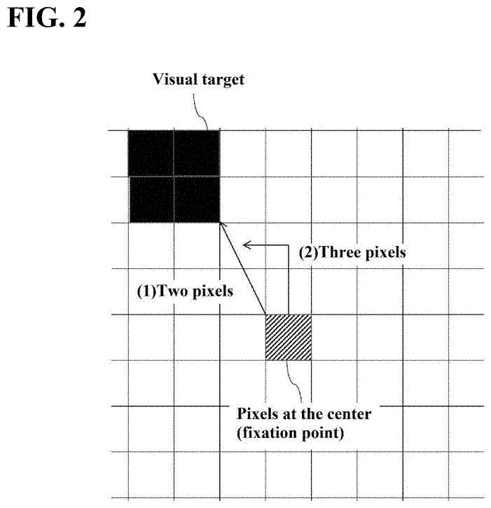

FIG. 2 is a diagram illustrating how to count the number of pixels from the pixel at the center of the touch screen of the vision testing device of this embodiment.

An example of how to count “the number of pixels from the pixel at the center of the touch screen to the pixels of the visual target” is to count the number of pixels on the shortest line from the pixel at the center of the touch screen to the pixels including the center of the visual target displayed on the touch screen ((1) in FIG. 2), or to add up the number of pixels from the pixel at the center of the touch screen to the pixels including the center of the visual target ((2) in FIG. 2).

In order to increase the tap sensitivity, the tap judgment function imparted to the pixels of the visual target may be extended and imparted to the pixels surrounding the visual target as well. As the number of pixels from the pixel at the center of the touch screen to the pixels of the visual target is larger, the degree of extension in this event may be increased.

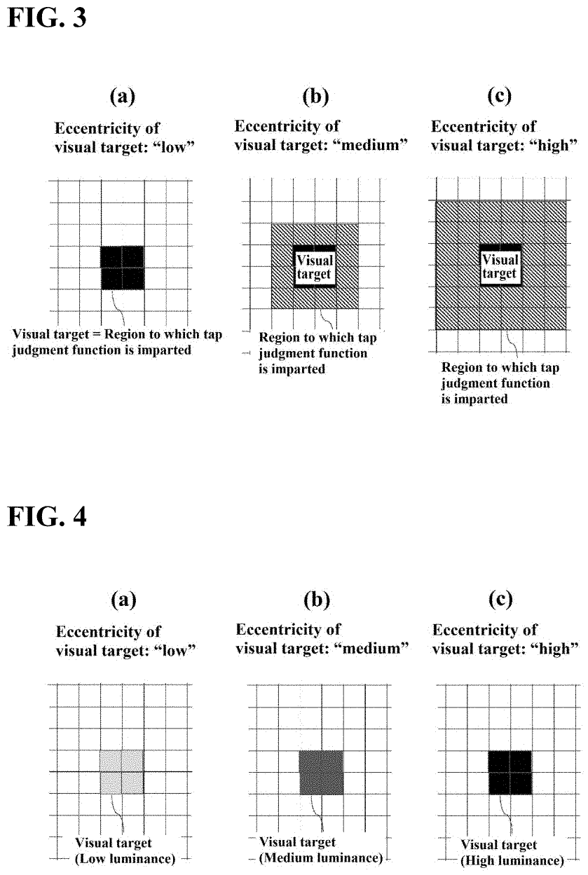

FIG. 3 is a diagram illustrating the arrangement of the visual target and the region to which the tap judgment function is imparted when the eccentricity of a visual target of the vision testing device of this embodiment is classified into three levels: low (FIG. 3(a)), medium (FIG. 3(b)), and high (FIG. 3(c)).

As a specific aspect of increasing the degree of extension, the range to which the tap judgment function is imparted may be defined by the number of pixels from the pixels of the visual target. For example, the number of pixels from the pixel at the center of the touch screen is classified into three levels: low, medium, and high. In a case of “low”, the tap judgment function may be imparted only to the pixels of the visual target, in a case of “medium”, the tap judgment function may also be imparted to a single circle of pixels surrounding the pixels of the visual target, and in a case of “high”, the tap judgment function may also be imparted to a double circle of pixels surrounding the pixels of the visual target.

An example using numerical values will be described. It is desirable that, in a case where the number of pixels corresponds to the eccentricity (visual angle) of about 20°, an area of the extended pixels to which the tap judgment function is imparted is about 4 times the area in a case where the number of pixels corresponds to the eccentricity (visual angle) of 10°, in consideration of the relation between the eccentricity (visual angle) and the visual acuity.

It should be noted that the present disclosure is not limited to these aspects, and there is no limitation on the number of pixels to be extended.

In addition to the above-described aspect, the following aspect may be adopted. For example, the tap sensitivity may be increased by decreasing the degree of pressure required to exert the tap judgment function imparted to the pixels of the visual target depending on the number of pixels from the pixel at the center of the touch screen. In other words, the pixels of the visual target displayed at the center of the touch screen does not exert the tap judgment function unless it is pressed firmly, while the pixels of the visual target displayed at the end of the touch screen can exert the tap judgment function only by a slight touch.

By adopting the present configuration, the difference in the tap sensitivity of the visual target on the touch screen between the central portion and the end portion can compensate for the difference in operability between the central portion and the end portion of the touch screen. As a result, the test accuracy when utilizing a touch screen can be improved.

### (Visual Target Attention Level)

One of the characteristics of this embodiment is to vary the attention level of the visual target depending on the eccentricity of the visual target from the fixation target. For the contents not described below, the description in (Tap sensitivity) can be applied.

The “attention level of the visual target” means a degree of ease for the testee to recognize the visual target. “To vary the attention level of the visual target” means to vary the degree of ease for the testee to recognize the visual target.

Specific examples of varying the attention level of the visual target include varying the contrast, luminance, or size of the visual target, or varying the presentation time of the visual target, or any combination thereof.

The phrase “eccentricity of the visual target from the center of the touch screen” means the degree of separation between the visual target and the center of the touch screen, from the center of the touch screen to the display limit of the touch screen or to the extreme end and farthest portion of the touch screen that is used for the vision test.

When using the touch screen, for the testee, it is easy to recognize the central portion of the touch screen while it is difficult to recognize the end portion of the touch screen. In contrast, by adopting the present configuration, the difference in the attention level of the visual target on the touch screen between the central portion and the end portion can compensate for the difference in ease of recognition between the central portion and the end portion of the touch screen. As a result, the test accuracy when utilizing a touch screen can be improved.

A specific example of providing a difference in the attention level between the central portion and the end portion will be described below. The present disclosure is not limited to the following specific example.

FIG. 4 is a diagram illustrating the difference in the luminance of the visual target when the eccentricity of the visual target of the vision testing device of this embodiment is classified into three levels: low (FIG. 4(a)), medium (FIG. 4(b)), and high (FIG. 4(c)).

As the eccentricity is larger, increasing the attention level of the visual target, thereby increasing contrast, luminance, or size of the visual target, or increasing presentation time of the visual target, or any combination thereof is preferred. In a case of increasing the luminance of the visual target to increase the attention level of the visual target, the luminance of the visual target B with medium eccentricity is increased compared to the visual target A with low eccentricity, as shown in FIG. 4. Similarly, the luminance of the visual target C with high eccentricity is increased compared to the visual target B with medium eccentricity.

An example using numerical values will be described. Regarding the contrast and the luminance, the contrast in a case where the eccentricity is 20 pixels is desirably higher than the contrast in a case where the eccentricity is 10 pixels by about 6 dB±4 dB (2 dB to 10 dB), in consideration of the relation between the eccentricity and the sense of human.

Regarding the presentation time, the presentation time in a case where the eccentricity is 20 pixels is desirably about 2±0.5 times (1.5 to 2.5 times) longer than that in a case where the eccentricity is 10 pixels.

In a case of increasing the contrast and/or size of the visual target, which is another specific example of increasing the attention level of the visual target, and/or in a case of increasing the presentation time of the visual target, the term “luminance” in the above paragraphs is substituted by the term “the contrast, size of the visual target and/or the presentation time of the visual target”.

### (Response Time Limit)

One of the characteristics of this embodiment is that the vision testing device has a touch screen which presents a visual target to a testee and lets him/her tap the visual target within a response time limit, and further the response time limit given to the testee may be variable for each round of the multiple rounds of presenting the visual target to the testee during the period from the beginning to the end of the test.

The “response time limit” is a time from when a visual target is presented to the testee to when the testee presses the visual target on the touch screen and is regarded as correct. When the testee fails to tap the visual target within this time period, it is regarded as incorrect. After this time has elapsed, another visual target may be immediately presented to the testee, or the previous visual target may be temporarily hidden and the other visual target may be presented to the testee after an interval.

The following is a list of the specific aspects in which the response time limit given to the testee is variable when presenting the visual target during the period from the beginning to the end of the test.

In the multiple rounds of presenting the visual target to the testee during the period from the beginning to the end of the test, the response time limit may be variable depending on the number of visual targets presented at once for each round.

In this aspect, the visual target is presented to the testee in multiple rounds. A case is assumed where the number of visual targets is one for the first presentation, while the number of visual targets is four for the second presentation, and the response time limit is uniform. Even when the testee can tap in time for the first presentation, he/she may fail to tap in time for the second presentation. Therefore, the response time limit for the second presentation is prolonged compared to that for the first presentation. An amount of time to be prolonged can be set as desired, but may refer to the response time required by the testee in the past (details will be described later).

As described above, there is no limitation on the number of visual targets presented at once. However, when the number of visual targets presented at once is more than one, there is a possibility that the testee fails to tap in time without applying this embodiment. However, by applying this embodiment, the response time limit can be set to an appropriate length. Moreover, since the response time limit is not uniformly prolonged, but is variable depending on the number of visual targets presented at once, the response time limit is not unnecessarily prolonged, and thus the period from the beginning to the end of the test is not unnecessarily prolonged.

The response time limit may be variable depending on the response time required by the testee in the past.

The “response time required by the testee in the past” may be the actual response time at the time when the visual target was presented for a vision test performed on a different date, or the actual response time required for the first or second presentation of the visual target during the period from the beginning to the end of the test.

In any case, in order to determine the degree to which the response time limit is prolonged in the aspect described above, namely, in the aspect in which the response time limit is variable depending on the number of visual targets presented at once, it is better to link the number of visual targets at that time to the actual response time, and save the linked data in the storage unit in the vision testing device, the cloud on the network, or the like. Then, based on the data, the degree to which the response time limit is prolonged depending on the number of visual targets presented at once may be determined.

A case will be described in detail where “the response time required by the testee in the past” is the actual response time required for the first or second presentation of the visual target during the period from the beginning to the end of the test.

The number of rounds of presenting the visual target to the testee during the period from the beginning to the end of the test is n or more (n is an integer of 3 or more), and the kth round (k is an integer of 2 or more, and k<n) of presenting the visual target is assumed. The number of visual target is assumed to be a fixed value of more than one. In that case, the response time limit may be set based on at least one of the actual response times required by the testee from the first to (k−1)^{th} round of presenting the visual target.

As an example, the average of the actual response time in the first round and the actual response time in the second round may be set as the response time limit or the response time limit with a margin of a few tenths of seconds added thereto, in the third and subsequent rounds. Of course, the present disclosure is not limited to this example and may, for example, refer only to the actual response time in the first round instead of the average, or to the average of the actual response time in the second round and the actual response time in the third round.

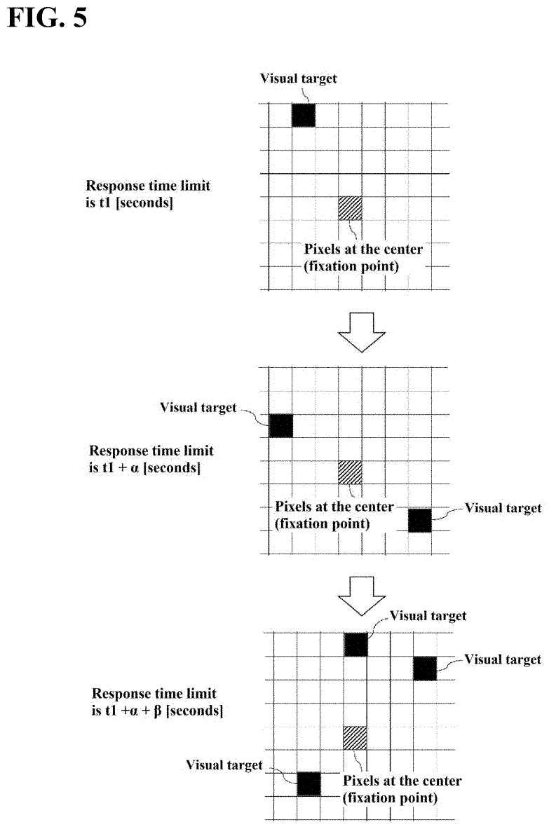

FIG. 5 is an explanatory diagram illustrating how the response time limit is varied depending on the number of visual targets displayed at once in the vision testing device of this embodiment.

For example, a case is assumed where the number of visual targets is one for the first presentation of the visual target, the number of visual targets is two for the second presentation, and the number of visual targets is three for the third presentation, and when the first response time limit is t1 [seconds], the second response time limit may be t1+α [seconds], and the third response time limit may be t1+α+β [seconds].

An example using numerical values will be described.

The first response time limit is 2500 [msec], and the response time limit is increased by 1000 [msec] for each additional visual target. Thereafter, when the response time until the third round falls below 1500 [msec], it is conceivable to set the response time limit to 2000 [msec] and the increment due to the number of visual targets presented to 800 [msec]. Thereafter, the amount of variation due to the response time limit and number of visual targets presented shall be increased or decreased in accordance with the response time.

A specific example of the configuration of the vision testing device will be described below. The present disclosure is not limited to the following specific example.

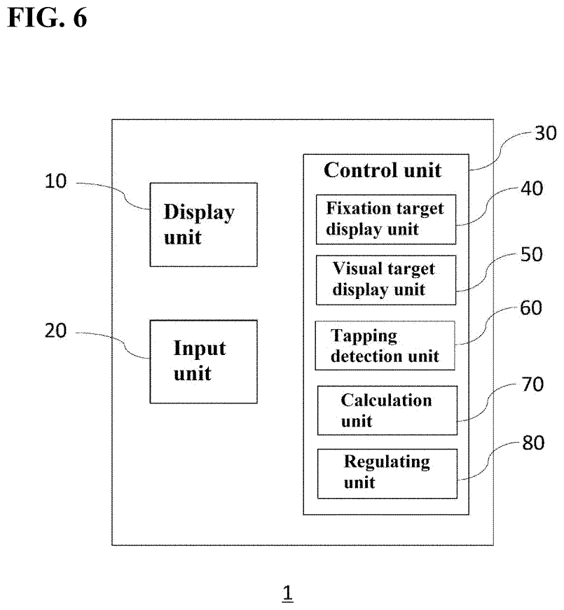

FIG. 6 is a block diagram including a configuration of a control system of the vision testing device of this embodiment.

The tablet device 1, which is a computer, may include a display unit 10, an input unit 20, and a control unit 30. It is preferred that the control unit 30 executes a predetermined program, so that the tablet device 1 functions as a fixation target display unit 40, a visual target display unit 50, a tapping detection unit 60, a calculation unit 70, and a regulating unit 80.

The display unit 10 is a portion displaying the fixation target and the visual target to present them to the testee, which is a so-called display. The input unit 20 is a portion accepting that the testee has tapped the visual target, which is a so-called touch screen. In a case of a tablet device, the touch screen itself serves as the display.

The control unit 30 realizes various functions (means) during the visual field test. As the control unit 30, the control unit mounted in the known tablet device may be utilized.

As the control unit 30, a combination of CPU (Central Processing Unit), RAM (Random Access Memory), ROM (Read Only Memory), HDD (Hard Disk Drive), various interfaces and the like is mounted in a computer (hereinafter, a computer indicates a tablet device). Further, the control unit 30 is configured so that the CPU realizes various functions by executing a predetermined program stored in the ROM or the HDD. The predetermined program for realizing each function is installed in a computer and used. However, the program may be provided by being stored in a computer readable storage medium, or may be provided through a communication line connected to the computer.

The control unit 30 includes, as an example of functions (means) realized by executing the above-mentioned program, the fixation target display unit 40, the visual target display unit 50, the tapping detection unit 60, the calculation unit 70, and the regulating unit 80.

The fixation target display unit 40 has a configuration to display the fixation target on the display unit 10. The visual target display unit 50 has a configuration to display the visual target on the display unit 10. The fixation target display unit 40 and the visual target display unit 50 may adopt a function of the existing tablet device to display an image on its touch screen.

The tapping detection unit 60 is a portion that detects tapping when the testee taps the visual target, and may adopt a pressure detection function of the existing tablet device.

The calculation unit 70 may be any one or a combination of the following:
a portion for calculating the degree of variation in the tap sensitivity depending on the eccentricity of the visual target;
a portion for calculating the degree of variation in the attention level depending on the eccentricity of the visual target; and
a portion for calculating the response time limit depending on the number of visual targets presented at once in each round.

Note that the calculation unit 70 may utilize a calculation function of the known tablet device.

The regulating unit 80 may be any one or a combination of the following:
a portion for reflecting the degree of variation in the tap sensitivity onto the touch screen;
a portion for reflecting the degree of variation in the attention level onto the touch screen; and
a portion for determining the response time limit adopted for the kth round of presenting the visual target to be performed, from the data (e.g., matrix) of the actual response time linked to the number of visual targets at that time, stored in the storage unit in the vision testing device, cloud on the network, or the like.

Note that the regulating unit 80 and the calculation unit may have common configuration.

The technical concept of this embodiment is not limited to a vision testing device which includes a touch screen as a part of its configuration. For example, each of the above-described functions may be exhibited by the vision testing system connected to the known tablet device via a wired or wireless network. Moreover, it can be said that the technical concept of the present disclosure is also reflected in the program and its storage medium which causes the computer (e.g., tablet device) to function so as to exhibit each of the above-described functions.

The test using the vision testing device may be performed in a state of binocular vision or in a state of monocular vision with one eye covered.

The technical scope of the present disclosure is not limited to the embodiment described above, but includes forms with various modifications and improvements as far as the specific effects obtained by the constituent features of the present disclosure and combinations thereof can be derived.

It is preferred to further include a mark for standby position of a hand of the testee to prevent the hand from remaining positioned between the testee and the touch screen.

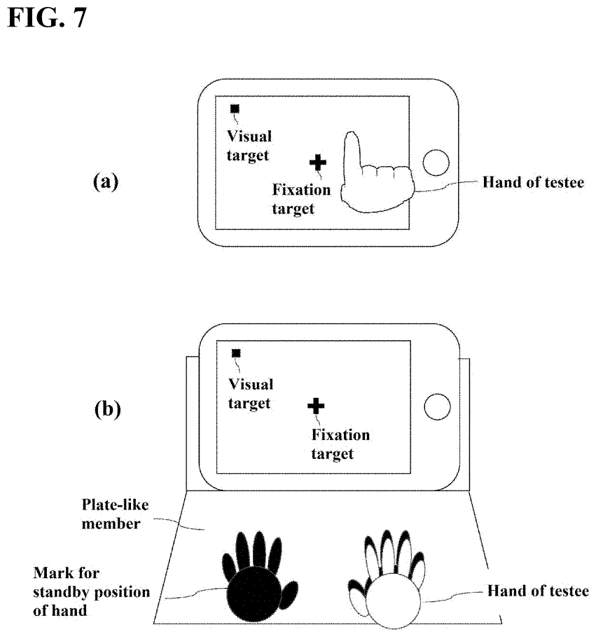

FIG. 7(a) is a diagram illustrating a case where a hand of the testee remains positioned between the testee and the touch screen when the visual target is presented to the testee in the vision testing device of this embodiment, and FIG. 7(b) is a diagram illustrating a case where a mark for standby position of a hand is provided and the testee complies with the mark.

As illustrated in FIG. 7(a), in a case where the multiple rounds of presenting the visual targets to the testee is performed by the vision testing device of this embodiment, there is a possibility that the testee forgets to place his/her hand back into place, and the hand remains positioned in the vicinity of the touch screen. As a result, the response time is shortened, which may have effect on the actual test result. In order to prevent it, the mark for standby position of a hand is provided as illustrated in FIG. 7(b) to facilitate the testee to place his/her hand back to the marked position.

Moreover, a chin support (not shown) on which the testee rests his/her chin may be provided to maintain a positional relation between the touch screen and the eye of the testee.

The shape of the mark for standby position of a hand is arbitrary. It may be in the shape of a hand, or may be simply a straight line or the like. The mark for standby position of a hand may be placed on a separately prepared plate-like member. In that case, an edge of the plate-like member may be bent to serve as a stand that maintains the tilted position of the tablet device. In that case, the tilt angle of the tablet device can be fixed. In addition, a chin support can be provided on the plate-like member to fix the positional relation between the eye of the testee and the touch screen of the tablet device.

In the above embodiment, a tablet-type testing device is described as an example of the testing device. However, the vision testing device is not limited to this example, and may be applied, for example, to a testing device that displays test images on a display equipped with a touch screen for a visual field test.

Moreover, setting the degree of variation in the tap sensitivity, the degree of variation in the visual target attention level, and/or the degree of variation in the response time limit in advance would eliminate the calculation unit.

### LIST OF REFERENCE NUMERALS

1 . . . Tablet device
10 . . . Display unit
20 . . . Input unit
30 . . . Control unit
40 . . . Fixation target display unit
50 . . . Visual target display unit
60 . . . Tapping detection unit
70 . . . Calculation unit
80 . . . Regulating unit

1. A vision testing device comprising a touch screen, sequentially displaying visual targets on the touch screen while displaying a fixation target whose position is fixed on the touch screen, during a period from the beginning to the end of the test, and providing a test result based on whether a testee taps the visual target or not.

2. The vision testing device according to claim 1, wherein multiple rounds of presenting the visual target to the testee include a round where the number of visual targets to be presented at once is more than one, during the period from the beginning to the end of the test, and the testee tapping all of the visual targets presented is considered as correct.

3. The vision testing device according to claim 1, further comprising a mark for standby position of a hand of the testee to prevent the hand from remaining positioned between the testee and the touch screen.

4. A vision testing system sequentially displaying visual targets on a touch screen while displaying a fixation target whose position is fixed on the touch screen, during a period from the beginning to the end of the test, and providing a test result based on whether a testee taps the visual target or not.

5. A vision testing program for making a computer function to sequentially display visual targets on a touch screen while displaying a fixation target whose position is fixed on the touch screen, during a test, and to provide a test result based on whether a testee taps the visual target or not.

6. The vision testing device according to claim 2, further comprising a mark for standby position of a hand of the testee to prevent the hand from remaining positioned between the testee and the touch screen.
